(19) United States
(12) Patent Application Publication
(10) Pub. No.: US 2010/0196456 A1
(43) Pub. Date: Aug. 5, 2010

(54) EMULSION PRECONCENTRATES AND MICELLAR FORMULATIONS CONTAINING WOOD RESINS

(76) Inventor: Daniel Strasser, Gossau (CH)

Correspondence Address: LADAS & PARRY LLP, 26 WEST 61ST STREET, NEW YORK, NY, 10023, US

(21) Appl. No.: 12/598,931

(22) PCT Filed: May 6, 2008
(86) PCT No.: PCT/CH08/00207

§ 371 Date: Mar. 8, 2010

(30) Foreign Application Priority Data
May 9, 2007 (CH) 757/07

(51) Int. Cl. A61K 9/127; A61K 38/43; A23L 1/30; A23L 1/00

(52) U.S. Cl. 424/450; 424/94.1; 426/72; 426/89

(57) ABSTRACT

The invention relates to micelles and emulsion preconcentrates comprising i) one or more active ingredients and ii) wood resin, optionally in combination with gum arabic, and iii) optionally additional adjuvants; products containing said micelles and/or emulsion preconcentrates, method for the production of micelles, emulsion preconcentrates, and products.

This application claims the priority of Swiss patent application 757/2007 and its entire disclosure, which was submitted on 9 May 2007, to which reference is hereby made.

The invention relates to micelles and emulsion pre-concentrates containing i) one or more active ingredients and ii) wood resin, optionally in combination with gum arabic as well as iii) optional adjuvants; products containing these micelles and/or emulsion pre-concentrates, a process for the production of micelles, emulsion pre-concentrates and products.

A typical problem in the use of active ingredients is insufficient bioavailability. Bioavailability is understood to mean the portion of unmodified active ingredient that is available in the circulatory system of humans, animals, and plants. It indicates how quickly and the extent to which the active ingredient is reabsorbed and made available at the site of action. For active ingredients administered intravenously (by IV), the bioavailability is by definition 100%. The absolute bioavailability indicates the bioavailability of a substance administered in any manner (e.g. peroral) compared to intravenous administration. The bioavailability observed after oral administration is also called oral bioavailability. Improving oral bioavailability is of particular importance, because oral administration of active ingredients is preferred over other forms of administration.

It is known that the bioavailability of active ingredients can be improved by adding adjuvants. Such compositions containing active ingredient(s) and adjuvant(s) are often characterized as “formulations” or “active ingredient formulations.” It is further known that wood resin (E445, a known natural substance/natural substance mixture) is used in foodstuffs and in formulating pharmaceuticals. The known formulations may, depending on the type, affect solubilization, micellation, and/or dispersion of active ingredients. In many cases, however, the results are unsatisfactory or only somewhat satisfactory.

With numerous active ingredients, the effectiveness of which is known, the application makes for difficulties, because their solubilization and stabilization are difficult. An example of such an active ingredient would be coenzyme Q10. The possibility of solubilizing and simultaneously stabilizing in micelles a fat-soluble active ingredient of this or similar quality with the same or even increased loading capacity would open up undreamed-of prospects. Thus, active ingredients treated in this manner would no longer simply be administered orally as capsules, but would rather be mixed into the active ingredients of a beverage, for example a sports drink. Making available targeted, previously fat-soluble active ingredients in beverages would bring about great advantages for users, because these active ingredients would be soluble in said beverages, and moreover would remain stable. In addition to medicinal active ingredients, there is a whole host of additional active ingredients, which, until now, it has not been possible to solubilize, disperse, or stabilize, or to do so only unsatisfactorily.

In addition to insufficient bioavailability (and/or general solubility), other problems include insufficient stability (shelf life), unfavorable visual characteristics (such as turbidity and/or discoloration), unfavorable physiological properties (such as side effects of the adjuvants, odor, taste), and/or difficulty in producing the formulation and/or the products containing them.

Thus, there is a need to provide formulations and production methods for active ingredients, which would overcome one or more of the disadvantages mentioned. There is a particular need for adjuvants that will initiate, enable, support, or facilitate solubilization, micellation, and dispersion. It would be ideal if such adjuvants were approved (i.e. already in the pharmaceutical or foodstuff lists or comparable registers), because they are classified as harmless and no objections or reservations would be expected from the health authorities, consumer organizations, or other organizations with a vested interest. However, it is not only the mere solubilization and dispersion of an active ingredient that is important; it is also important to stabilize an active ingredient (optionally solubilized and/or micellized) to provide/improve the longest and most complete effect and/or reabsorption.

Thus, the present invention relates to an improved process for solubilizing, dispersing, micellizing, and, if necessary, stabilizing active ingredients, thereby providing products which would solve one or more of the aforementioned problems. These active ingredients include, for example, lipophilic active ingredients in a hydrophilic environment as well as hydrophilic substances in a lipophilic and hydrophilic environment, particularly in an aqueous environment. The solubilization, dispersion, and micellation of active ingredients, as described herein, will also achieve stabilization of said ingredients in a hydrophilic environment. Finally, the invention also comprises the use of said types of substances and active ingredients. The present invention further relates to active ingredients which are contained in micelles, emulsion pre-concentrates containing such micelles, and products containing micelles or emulsion pre-concentrates.

The objects outlined above are achieved according to the independent claims. The dependent claims represent advantageous embodiments. The general, preferred, and especially preferred embodiments and areas indicated in the context of the present invention may be combined with one another in any manner. Likewise, individual definitions and embodiments may be omitted and/or may not be relevant. In the following, various aspects of the invention are explained in detail.

In a first aspect, the invention relates to micelles with a diameter of from 2 to 300 nm, containing i) one or more active ingredients, ii) wood resin, iii) optionally one or more adjuvants. Surprisingly, it was found that active ingredients which are embedded in such micelles have a significantly improved bioavailability.

In an advantageous embodiment, the invention relates to micelles with a diameter of from 2 to 300 nm, containing i) one or more active ingredients, ii) wood resin and gum arabic, iii) optionally one or more adjuvants.

The invention is explained in further detail in the following, wherein, in particular, the individual components are described in further detail.

Component i) The term “active ingredient” in the context of the present invention is understood to be a natural, nature-identical, or synthetic compound, which brings about a physiological effect in/on humans, animals, or plants. According to their function, active ingredients can be subdivided into groups of foodstuff additives, active pharmaceutical ingredients (for humans or animals), active cosmetic ingredients, and active plant ingredients, wherein individual active ingredients may also be allocated to multiple groups. In connection with the present invention, active ingredients which are difficult to formulate in another manner and/or which have insufficient bioavailability are preferred. Such active ingredients typically have a water-solubility of less than 10 g/L (20° C.; neutral medium) or less than 100 g/L (20° C., physiological medium, e.g.

synthetic bodily fluid). The molar mass of such active ingredients can vary within a wide range and comprises “small molecules” starting from a molar mass of about 50 g/mol to macromolecules of about 50,000 g/mol.

The term “food additives” comprises those compounds which are approved for human consumption and which may be added to foodstuffs. Food additives are known to a person skilled in the art and can be identified, for example, in the EDI [Swiss Federal Department of Domestic Affairs] Directive on Additives Permissible in Foodstuffs (Directive on Additives, ZuV) of 23 Nov. 2005. In the scope of the present invention, particular reference is made to the class of vitamins (e.g. vitamins A and E), fatty acids (e.g. unsaturated fatty acids such as omega-3 fatty acids and omega-6 fatty acids), enzymes (e.g. from the group of flavinoids), coenzymes (e.g. coenzyme Q10), and pigments (e.g. carotenoids such as beta carotene, lycopene, and lutein).

The term “active pharmaceutical ingredients” comprises those compounds which are approved for the prevention and treatment of human or animal illnesses. Active pharmaceutical ingredients are known to a person skilled in the art and can be identified, for example, in the “Orange Book” or the “Red List.”

The term “active cosmetic ingredients” comprises those compounds which are used in cosmetics and are responsible for a physiological effect (e.g. on the skin/hair/nails) or a visual effect (e.g. coloring). Active cosmetic ingredients are known to a person skilled in the art and can be identified, for example, according to the INCI. In the scope of the present invention, particular reference is made to the class of cosmetics.

The term “active plant ingredients” comprises those compounds which are used in the area of agriculture, forestry, and landscaping. Active plant ingredients are known to a person skilled in the art and can be identified, for example, in the “Pesticide Manual.” In the scope of the present invention, reference is made to the group of herbicides, fungicides, insecticides, and growth regulators.

Reference is made to the fact that active ingredients can also fall into more than one of the foregoing categories, for example, food additive and pharmaceutical (e.g. because the pharmaceutical effect is not yet known or is disputed). In the scope of the present invention, it is sufficient if a person skilled in the art can allocate an active ingredient, as previously defined, to at least one of these classes.

Component ii) Compounds of group ii) can be combined under the generic term “micelle-forming substances.” As previously shown, the micelle according to the invention can contain as component ii) either wood resin or a mixture of wood resin and gum arabic. The use of wood resin generally ensures stable and quick micelle formation. However, for certain applications, it may be advantageous if the micelle formation is initiated and/or supported by the further addition of gum arabic.

The term “wood resin” is understood to mean the glycerol ester of the known natural product, which may be obtained as an extract from conifer root stock. In particular, “wood resin” indicates the commercially available compound E445. Due to its natural origin, the composition of the wood resin may vary, wherein the different mixtures are included in the various purity grades (“natural wood resin”). Furthermore, the term “wood resin” also refers to glycerol esters of resin acids, which are available as a synthetic or a semi-synthetic (“synthetic wood resin”). “Resin acids” are known to a person skilled in the art; in particular, unsaturated carboxylic acids of the empirical formula C₂₀H₃₀O₂ are included in this term.

The term “gum arabic” refers to the known, commercially obtainable natural substance, which is registered as E414 or CAS 9000-01-05. Due to its natural origin, the composition may vary, wherein the different mixtures are included in the various purity grades (“natural gum arabic”). Furthermore, the term includes poly-arabic acid and its derivatives, particularly acidic alkaline earth and alkaline salts, which are obtainable by synthetic or semi-synthetic means (“synthetic gum arabic”).

Component iii) The term “adjuvants” is understood to mean those compounds that have a positive influence on the micelle, the emulsion pre-concentrate, and/or the finished product. A group of adjuvants are dissolving agents that reduce the melting point of wood resin or that form a homogenous phase with wood resin below its melting point. Typical examples are compounds from the group of polyols such as, for example, glycerol, propylene glycol, polyethylene glycol 400, etc. Such adjuvants are preferably added when the active ingredient to be processed is thermally unstable at the melting point of the wood resin (e.g. proteins).

The term “micelle” is known to a person skilled in the art. It characterizes structures with a “core” and a “shell” in a certain order of magnitude and a form described as an irregularly shaped ellipsoid or sphere. Micelles typically have a diameter of less than 500 nm and have a certain size distribution resulting from their production. Size and size distribution can be determined by optical methods (e.g. microscope). In the scope of the present invention, micelles preferably have a diameter of from 2-300 nm, with 10-100 nm being particularly preferred. Typically, at least 66%, or preferably 75%, or most preferably 80% of the micelles are within the aforementioned range. The core of the micelle essentially contains the active ingredient or ingredients; the shell essentially contains wood resin or wood resin and gum arabic. Because a micelle is a dynamic structure, the transitions between the core/shell and the shell/environment are sometimes more or sometimes less sharply differentiated or blurred. Any additional adjuvants present are either primarily in the core or primarily in the shell of the micelle, depending on their coefficient of distribution. It is further known that the aforementioned core of the micelle is not present in a particulate form, i.e. not as a solid.

Further advantageous embodiments are explained in more detail in the following:

In one advantageous embodiment, the present invention relates to micelles, as previously described, containing one or more active ingredients with a solubility of less than 1 g/L, selected from the group of food additives, active pharmaceutical ingredients, active cosmetic ingredients, and active plant ingredients.

In a further advantageous embodiment, the present invention relates to micelles, as previously described, in which the active ingredient is coenzyme Q10.

In a further advantageous embodiment, the present invention relates to micelles, as previously described, in which the active ingredient is selected from the group comprising i) one or more omega-3 and/or omega-6 fatty acids; ii) one or more polyphenols, preferably from the group of flavonoids, particularly curcumine or catechins; iii) one or more carotinoids, particularly lutein and/or beta carotene; iv) one or more vitamins from the E group (tocopherols and tocotrienols) particularly alpha tocopherol); v) one or more vitamins from the B group, particularly biotin and/or choline.

In a further advantageous embodiment, the present invention relates to micelles, as previously described, containing one or more adjuvants selected from the group of polyols.

In a further preferred embodiment, the present invention relates to micelles, as previously described, in which the ratio of compounds of group i) (active ingredient(s)) to compounds of group ii) (wood resin or wood resin+gum arabic) is in a range of from 20:1 to 1:10% by mass, with 10:1 to 1:1 being preferred. It is generally desirable to minimize the quantity of compounds from group ii), particularly wood resin, and to maximize the quantity of active ingredients. On the other hand, considering the targeted use, sufficient stability and bioavailability must also be present. The optimum ratio of active ingredient(s) to compounds from group ii) can be determined using a simple series of tests. The properties of the active ingredient, particularly its solubility, will influence the aforementioned ratio. In comparison to the known active ingredient formulations, the following method can be used to beneficially influence the ratio of active ingredient(s) to compounds of group ii) (particularly wood resin) while also achieving a narrow size distribution in the micelles. Thus, the micelles according to the invention are stable and enable a high level of bioavailability and a favorable release profile for the active ingredient. As previously mentioned, the wood resin can be replaced by a combination of wood resin and gum arabic. This enables one or more of the following effects to be achieved: a) reducing the content of wood resin, b) increasing the active ingredient load, c) improving visual properties of the end product (e.g. transparency). If a combination of wood resin and gum arabic is used, a portion of the wood resin can be substituted with the gum arabic. Advantageously, the ratio of wood resin to gum arabic is in a range of from 1:10 to 10:1, for Example 1:1.

In a second aspect, the invention relates to emulsion pre-concentrates containing i) a lipophilic phase, which contains micelles as previously described, and ii) an aqueous phase. Typically, micelles in and of themselves are not very resistant but are dispersed in a further phase. Such emulsion pre-concentrates typically accumulate immediately during production. Such emulsion pre-concentrates can be provided to other industries for further processing (for information on manufacturing products, see below) or can be further processed immediately. Accordingly, the use of emulsion pre-concentrates to manufacture products, as defined below, is also included.

Emulsion pre-concentrates can be characterized by the type of emulsion and by the micelle concentration, size, and size distribution. Type: Emulsion pre-concentrates can be characterized as oil in water (O/W; the homogenous phase is aqueous) or water in oil (W/O; the disperse phase is aqueous). O/W emulsions are preferred. Concentration: Typically, the micelle concentration is higher in emulsion pre-concentrates than it is in the products. For the present invention, a ratio of lipophilic phase to aqueous phase ranging from 1:10 to 1:0.5 has proven to be favorable. The size and size distribution of the micelles was previously explained in conjunction with the description of the micelle, to which express references is hereby made.

Thus, in a preferred embodiment, the present invention relates to an emulsion pre-concentrate, as previously described, in which the ratio of lipophilic phase to aqueous phase ranges from 1:10 to 1:1.

The “lipophilic phase” of the emulsion pre-concentrates comprises primarily, or preferably exclusively, micelles, as previously described.

In one embodiment, the “aqueous phase” comprises (only) water. Due to the thermodynamics, the aqueous phase always contains wood resin and an active ingredient and optionally also further adjuvants found in the micelle in a certain proportion; however, for the sake of simplicity, reference is made to a “pure” aqueous phase. The water used can be of various purity grades (e.g. purified, deionized, applications for IVs, etc.) depending on the further intended application. These purity grades are included in the invention.

In a further embodiment, the aqueous phases contain further components. Such components can serve to influence the pH value (acids, bases, buffers), to influence the ion strength (buffers, salts), or to influence the rheological properties (thickeners). One or more components may be added. Such components are known to a person skilled in the art and can be identified, for example, in Fiedler's Lexikon der Hilfsstoffe für Pharmazie, Kosmetik and angrenzende Gebiete (1989) [Encyclopedia of Adjuvants for Pharmaceuticals, Cosmetics, and Associated Industries] (1989).

In a further embodiment, the invention relates to an emulsion pre-concentrate, consisting of a transparent gel based on at least one wood resin and a micellated active ingredient solubilized therein, whose consistency is between a semisolid (aspic-like) and a liquid.

In a third aspect, the invention relates to products from the group of foodstuffs, cosmetics, pharmaceuticals, and plant protection agents containing micelles, as previously described, or one or more emulsion pre-concentrates, as previously described.

Such products can have the most varied of forms. The following products are listed for illustrative purposes: i) liquids: as a beverage in the food industry; as a solution, drops, or syrup in the pharmaceutical industry; as a spray or solution in the cosmetics industry; as a spray solution in the agriculture industry; ii) gels or jellies in baked goods (as a spread for bread); as creams to smooth on the body (pharmaceuticals, cosmetics); iii) creams or pastes in baked goods, as a spread for bread, in snacks; as crèmes (pharmaceuticals, cosmetics); iv) capsules or coated pills, as an efficient and anhydrous formulation for the pharmaceutical industry or the food industry; v) coated solid bodies, as an improved formulation for snack products, cereals, or grain preparations in the food industry. As the previous list makes clear, the product can be the finished market product (e.g. liquids) or part of a market product (e.g. the cream in a baked good).

The emulsion pre-concentrate can be added to the products, as described previously, in different quantities. The quantity depends, among other things, on the desired results/the required quantity of active ingredient added. This quantity can be determined in a simple series of tests. Because the emulsion pre-concentrates ensure improved bioavailability, generally a low quantity of emulsion pre-concentrate can be processed, which is considered to be a significant advantage. It has been shown that, due to their properties, emulsion pre-concentrates can be added to practically any product without negatively influencing the properties of the product. Thus, liquids remain essentially clear and stable for a long time, which, from the perspective of foodstuff marketing, is considered to be a significant advantage.

In a preferred embodiment, the present invention relates to a product from the group of foodstuffs, particularly a beverage containing coenzyme Q10.

In another preferred embodiment, the present invention relates to a product from the group of foodstuffs, particularly beverages containing tocotrienol, beta carotene, lutein, or catechins (one or more).

In a further embodiment, the invention relates to a product containing wood resin and a micellated active ingredient solubilized, dispersed, and/or stabilized therein, wherein the consistency of the product is between a semisolid and a liquid.

In a further embodiment, the present invention relates to a product, comprising a transparent gel, which contains i) wood resin and ii) one or more solvents from the group of water, glycerin, propylene glycol, polyethylene glycol 400, ethanol, macrogol 400, isopropanol, as well as one or more lipophilic or hydrophilic active ingredients solubilized, dispersed, and stabilized therein.

The consistency of the products can vary to a great extent. Thus, products included are solid (firm enough to cut, brittle), semi-solid (e.g. aspic-like), and liquid (thin liquid such as water or syrup). The products do not have to be homogenous (e.g. in creams, foams) or can be encapsulated (e.g. in coated pills).

In a fourth aspect, the invention relates to a process for producing an emulsion pre-concentrate, as previously described. In variant A, the invention relates to a process comprising the following steps: i) production of a homogenous phase either by melting wood resin at 30-110° C., or preferably at 40-60° C., or by dissolving wood resin in one or more adjuvants at 0-100° C., or preferably 0-60° C.; ii) portioned addition of an active ingredient, which is optionally dispersed in an aqueous phase, to a homogenous phase by stirring; iii) optional further addition of an aqueous phase to the resulting stirred reaction mixture, wherein an aqueous phase is added during at least one of the reaction steps and wherein the temperature of the active ingredient, which is optionally dispersed in water, is within +/−10° C. of the homogenous phase.

In variant B, the invention relates to a process comprising the following steps: i) production of a homogenous phase either by melting wood resin and gum arabic at 30-130° C., or preferably at 40-60° C., or by dissolving wood resin and gum arabic in one or more adjuvants at 0-100° C., or preferably 0-60° C.; ii) portioned addition of an active ingredient, which is optionally dispersed in an aqueous phase, to a homogenous phase by stirring; iii) optional further addition of an aqueous phase to the resulting stirred reaction mixture, wherein an aqueous phase is added during at least one of the reaction steps and wherein the temperature of the active ingredient, which is optionally dispersed in water, is within +/−10° C. of the homogenous phase.

In variant C, the invention relates to a process comprising the following steps: i) production of a homogenous phase by melting wood resin and optionally gum arabic at 30-130° C., or preferably at 40-60° C.; ii) portioned addition of an active ingredient to a homogenous phase by stirring; iii) further addition of an aqueous phase to the resulting stirred reaction mixture, and wherein the temperature of the aqueous phase is +/−10° C. of the homogenous phase.

Thus, the invention relates to a process for solubilization, dispersion, micellation, and stabilization of active ingredients; emulsion pre-concentrates and products solubilized, dispersed, micellized, and stabilized according to the process; as well as the use of such emulsion pre-concentrates and products.

The term “solubilization” in the context of the present invention is understood to mean the conversion of a lipophilic substance into a hydrophilic substance.

The term “dispersing” in the context of the present invention is understood to mean the blending of different types of substances under a shear force.

The term “micellation” in the context of the present invention is understood to mean the conversion of active ingredient into micelles.

The term “stabilization” in the context of the present invention is understood to mean a process which creates a system in which an active ingredient remains unchanged over a longer time frame (particularly 1 day to 1 year, preferably 5-200 days).

It is understood that the process mentioned in this description relates to both the production using wood resin as well as the production using wood resin and gum arabic. Reference to wood resin alone at various places is simply for the sake of readability.

The previously described processes are thus suitable for solubilizing, dispersing, and stabilizing an active ingredient(s) in micelles, which is characterized in that
i) wood resin or wood resin/gum arabic and an active ingredient are intimately dispersed;
ii) the homogenous phase formed is subsequently covered with water of the same temperature, and iii) the gel which is thereby spontaneously formed is homogenized.

In an advantageous embodiment, the temperature of the aqueous phase is within +/−10° C. of the homogenous phase.

The invention relates, in particular, to a process, as previously described, characterized in that the aqueous phase comprises water.

The invention relates, in particular, to a process, as previously described, characterized in that the adjuvants are selected from the group of polyols.

In a further embodiment, the present invention relates to a process for solubilizing, dispersing, micellizing, and stabilizing active ingredients, in which wood resin is used and the active ingredient to be treated is intimately dispersed in suspension, which up to that point has been fixed, wherein said suspension is subsequently covered with water of the same temperature and the gel that is thereby spontaneously formed is homogenized.

In a further embodiment, the present invention relates to a process for solubilizing, dispersing, micellizing, and stabilizing active ingredients, as previously described, characterized in that the wood resin is melted and the active ingredient to be treated is intimately dispersed into this suspension, wherein, after the substance to be treated is added to the dispersed substance, said dispersed substance is covered with water and the entirety is once again homogenized as a unit.

In a further embodiment, the present invention relates to a process for solubilizing, dispersing, micellizing, and stabilizing active ingredients, as previously described, characterized in that wood resin is dissolved in an adjuvant at room temperature, the temperature of the dispersed substance is maintained, the active ingredient is also dispersed, the melt is covered with water of the same temperature, and the melt is homogenized, whereby a lightly turbid to transparent gel results.

In a further embodiment, the present invention relates to a process for solubilizing, dispersing, micellizing, and stabilizing active ingredients, as previously described, characterized in that wood resin is dissolved at room temperature, the mixture of substances is reduced by adding one or more adjuvants comprising the group of water or glycerin at the same temperature, then the active ingredient to be solubilized, which is thermally unstable at best, is dispersed into the resulting melt, the melt is covered with water of the same temperature, and the melt is homogenized, whereby a transparent gel results.

As is clear from the previous explanations, proper temperature control is beneficial for the process. The expressions “same temperature” and “constant temperature” are understood to mean essentially the same temperature. This can vary depending on other process parameters, the design of the equipment, and the substances used. Typically, a temperature range of +/−10° C. is advantageous, with a temperature range of +/−5° C. being particularly advantageous. Correspondingly, room temperature is considered to be 22° C. +/−10° C., or preferably +/−5° C.

A further important process step has proven to be that the wood resin melt, along with the active ingredient dissolved therein, is covered with a sufficiently deep layer of water of approximately the same temperature. This measure results in the immediate self-formation of a transparent gel under the water. Without this covering with water of the same temperature, the melt hardens and cannot be applied or is very difficult to apply in this form. Therefore, the melt should be covered while it is still in liquid form with water of about the same temperature. The gel will also form with cold water, but then primarily a dispersion of the active ingredient results. After the water of the same temperature has been added and a cover has been formed over the melt (namely, the water flows over the melt), gel formation begins and the gel quickly swells upward toward the surface of the water by the same amount as the water it absorbs.

This gel formation observed from the exterior is supported by the quick mixing of water and melt, for example through targeted stirring. The gel takes on a micellar structure and has the consistency of a very fine solution. Gels with a droplet diameter of less than 40 nm may be obtained in this manner, which do not refract light and are therefore clearly transparent. This is all the more astounding because, for example, 10% of a fat-soluble active ingredient and about 10-20% wood resin are contained. These smallest fat droplets remain thermally stable, which means that even when the gel is heated, this does not result in any clustering of the fatty droplets and the micellar structure is also unchanged by the addition of water. The consistency is syrupy or thinner.

The gel is homogenized by stirring and diluted to a suitable viscosity by adding water or water/solvent mixtures. However, homogenization with great shear force is detrimental to gel formation. The resulting gel is then not transparent, which means that, in addition to solubilizing, dispersion also takes place. However, if stirring is carried out with normal blades, for example with a Stefan mixer, which has a rotating axle protruding vertically from the base of the container, with sharp perpendicularly arranged blades, continuously cutting into the mass being stirred, or with a dissolver mixer, this very quickly results in an optically purer, more attractive, and more transparent gel. In a gel such as this, microbial contamination takes place significantly more slowly than it does in a liquid.

The process for solubilizing and stabilizing an active ingredient is explained in more detail in the following: It has been shown that it does make a difference whether i) only wood resin is added in an aqueous phase and then an active ingredient is mixed in (in the hope that homogenization of the mixture will protect the mixture and stabilize it) or ii) wood resin and active ingredient are combined in a targeted manner. Without being held to one theory, it is considered to be particularly important that the wood resin and the active ingredient to be treated are combined on a molecular basis. With simple mixing according to i), the effect of the solubilizing, dispersing, and stabilization remains at only a few percent of the added active ingredient.

If wood resin is not contaminated with solvents, it remains liquid at room temperature. It has been shown that the majority of the lipophilic and hydrophilic active ingredients form a homogenous phase with wood resin. Without being held to one theory, this type of stabilization (in which wood resin is combined with the active ingredient to be solubilized on a molecular or quasi-molecular basis) is considered to be an important element for improving the ratio of wood resin to active ingredient. The result is that a lot of active ingredient can be encapsulated with much less wood resin. This also suppresses side effects from the adjuvants, because adjuvants are often added to the actual active ingredient in quantities that are somewhat excessive to extremely excessive.

In a fifth aspect, the invention relates to a process for producing a product, as previously described, characterized in that an emulsion pre-concentrate, as previously described, is mixed with further components of the product. The emulsion pre-concentrate can be added at various stages in the production of the product. The emulsion pre-concentrate can be added and distributed in the product according to the known methods and using the available devices. This flexibility is considered to be an advantage. When producing beverages, this can, for example, be the final production step. In general, technical considerations play an important role in the selection of a suitable production process for the product. Although the micelles and emulsion pre-concentrates according to this invention are temperature-stable, it has proven to be advantageous to avoid exposing the emulsion pre-concentrate to high temperatures for long periods of time.

The Examples shown in the following are intended to illustrate the invention without limiting it in any manner.

### EXAMPLE 1

100 g E445, 150 g E414, 50 g coenzyme Q10, and 16.65 g vitamin E are provided and heated to 105° C. for 1 hour. 186.35 g ultra-pure water at 95° C. is added within 10 min. The reaction mixture is mixed for 5 minutes at 92° C., degassed at this temperature, and filtrated. 500 g of a clear, stable liquid is obtained. Turbidity value: 5 FTU (Formazine Turbidity Unit).

### EXAMPLE 2

The procedure is similar to Example 1; the quantities of the components used as well as the properties of the micellar solution obtained are shown in the following table; vitamin E has been omitted.

| | Example 2.1 | Example 2.2 | Example 2.3 | Example 2.4 |
|---|---|---|---|---|
| Q 10 | 2% | 5% | 10% | 20% |

Comparison

| | | | | |
|---|---|---|---|---|
| E414 | Clear/opaque | Clear/opaque | Transparent | Turbid |
| (30%) | quick- | quick- | quick-dissolving | quick- |
| | dissolving | dissolving | 50 FTU | dissolving |
| | 15 FTU | 20 FTU | | 50 FTU |

According to the invention

| | | | | |
|---|---|---|---|---|
| E445 | Clear/ | Clear/ | Clear/opaque | Opaque/turbid |
| (20%) | transparent | transparent | slow-dissolving | slow-dissolving |
| | quick- | quick- | 15 FTU | 35 FTU |
| | dissolving | dissolving | | |
| | 2 FTU | 5 FTU | | |
| E414 | Clear/ | Clear/ | Clear/transparent | Clear/opaque |
| (15%) + | transparent | transparent | quick-dissolving | quick- |
| E445 | quick- | quick- | 5 FTU | dissolving |
| (10%) | dissolving | dissolving | | 15 FTU |
| | 2 FTU | 2 FTU | | |

Loading and the FTU value were used as an indicator for quality; the subjective dissolving rate was used as an indicator for manufacturability. It was shown that wood resin (E445) provides very good results as a micellizing agent; 5% solutions are high-quality and also easy to produce. Thus, these types of solutions as micellizing agents are superior to those with gum arabic (E414), because those are high-quality and easy-to-produce only up to a loading of 2%. Surprisingly, it has now been found that by combining gum arabic and wood resin as a micellizing agent, a particularly high loading, a low FTU value, and easy manufacturability can be achieved. Compared to gum arabic, this makes possible a tenfold loading with the same properties otherwise; compared to wood resin, this makes possible a fourfold loading (regarding manufacturability) and a twofold loading (regarding product quality).

### EXAMPLE 3

The procedure is similar to Example 1; the quantities of the components used as well as the properties of the micellar solution obtained are shown in the following table.

| Active | Example 3.1 | Example 3.2 | Example 3.3 |
|---|---|---|---|
| ingredient | 5% Flavinoids | 5% Lutein | 5% Beta carotene |
| E445 (20%) | Clear/transparent | Clear/transparent | cloudy/opaque |
| | slow-dissolving | slow-dissolving | slow-dissolving |
| | 10 FTU | 8 FTU | 10 FTU |
| E414 (15%) + | Clear/transparent | Clear/transparent | Clear/transparent |
| E445 (10%) | quick-dissolving | quick-dissolving | quick-dissolving |
| | 8 FTU | 6 FTU | 8 FTU |

It has been shown that a broad range of structurally very different active ingredients can be formulated.

Further embodiments, advantages, and applications of the invention result from the dependent claims and from the following description with figures. The

1. A composition in the form of micelles with a diameter of from 2-300 nm, containing i) one or more active ingredients, ii) wood resin, iii) optionally one or more adjuvants.

2. The composition according to claim 1, containing a mixture comprising wood resin and gum arabic as component ii), preferably in a ratio of from 1:10 to 10:1% by mass.

3. The composition according to claim 1, containing one or more active ingredients with a solubility of less than 1 g/L in each case, selected from the group of food additives, active pharmaceutical ingredients, active cosmetic ingredients, and active plant ingredients.

4. The composition according to claim 1, containing one or more adjuvants selected from the group of polyols.

5. The composition according to claim 1, in which the active ingredient is coenzyme Q10.

6. The composition according to claim 1, in which the active ingredient is selected from the group comprising:
i) one or more omega-3 fatty acids and/or omega-6 fatty acids;
ii) one or more polyphenols, preferably from the group of flavonoids, particularly curcumine;
iii) one or more carotinoids, particularly lutein and/or beta carotene; iv) one or more vitamins from the E group, particularly alpha tocopherol and/or tocotrienol; and/or v) one or more vitamins from the B group, particularly biotin and/or choline.

7. The composition according to claim 1, in which the ratio of active ingredient(s) to a compound of group ii) ranges from 20:1 to 1:10% by mass.

8. An emulsion pre-concentrate containing:
a) a lipophilic phase, which contains a composition according to claim 1, and
b) an aqueous phase.

9. The emulsion pre-concentrate according to claim 8, in which the ratio of the lipophilic phase to the aqueous phase ranges from 1:10 to 1:0.5.

10. A product from the group of foods, cosmetics, pharmaceuticals, and plant protection agents, which contains a composition according to claim 1 or an emulsion pre-concentrate containing a lipophilic phase, which contains a composition according to claim 1, and b) an aqueous phase.

11. The product according to claim 10, from the group of foodstuffs, which contains coenzyme Q10, tocotrienol, beta carotene, one or more catechins.

12. A process to produce an emulsion pre-concentrate according to claim 8, comprising the following steps:
Variant A, if only wood resin is used as component ii):
i) production of a homogenous phase either by melting the wood resin at 40-110° C. (preferably 40-60° C.) or by dissolving wood resin in one or more adjuvants at 0-100° C. (preferably 0-60° C.);
ii) portioned addition of an active ingredient, which is optionally dispersed in an aqueous phase, to the stirred homogenous phase;
iii) optional further addition of an aqueous phase to the resulting stirred reaction mixture, wherein an aqueous phase is added during at least one of the reaction steps and wherein the temperature of the active ingredient, which is optionally dispersed in water, is within +1-10° C. of the homogenous phase; or
Variant B, if a combination of wood resin and gum arabic is used as component ii):
i) production of a homogenous phase either by melting wood resin and gum arabic at 40-110° C. or by dissolving wood resin and gum arabic in one or more adjuvants at 0-100° C.;
ii) portioned addition of an active ingredient, which is optionally dispersed in an aqueous phase, to the stirred homogenous phase;
iii) optional further addition of an aqueous phase to the resulting stirred reaction mixture, wherein an aqueous phase is added during at least one of the reaction steps and wherein the temperature of the active ingredient, which is optionally dispersed in water, as well as the aqueous phase, is within +/−10° C. of the homogenous phase.

13. The process according to claim 12, characterized in that the aqueous phase comprises water.

14. The process according to claim 12, characterized in that the adjuvants are selected from the group of polyols.

15. The process for producing a product according to claim 10, characterized in that composition containing micelles with a diameter of from 2-300 nm, containing i) one or more active ingredients, ii) wood resin, iii) optionally one or more adjuvants, optionally in the form of an emulsion pre-concentrate wherein said components are present in a lipophilic phase and the pre-concentrate additionally contains an aqueous phase is mixed with further components of the product.
